(19) United States
(12) Patent Application Publication
(10) Pub. No.: US 2008/0004596 A1
(43) Pub. Date: Jan. 3, 2008

(54) DELIVERY OF AGENTS BY MICRONEEDLE CATHETER

(75) Inventors: Anthony Joonkyoo YUN, Palo Alto, CA (US); Kirk Patrick SEWARD, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Palo Alto Institute, Palo Alto, CA (US); Mercator MedSystems, Inc., San Leandro, CA (US)

(21) Appl. No.: 11/753,840

(22) Filed: May 25, 2007

Related U.S. Application Data
(60) Provisional application No. 60/803,130, filed on May 25, 2006.

(51) Int. Cl. A61M 31/00

(52) U.S. Cl. 604/508; 604/511

(57) ABSTRACT

Luminal diseases are treated by injecting palliative agents into tissue surrounding a target body lumen. A needle catheter may be placed in the target lumen and used to deliver the agent.

### CROSS-REFERENCES TO RELATED APPLICATIONS

This application claims the benefit of prior provisional application No. 60/803,130 (Attorney Docket No. 021621-002800US), filed on May 25, 2006, the full disclosure of which is incorporated herein by reference.

### BACKGROUND OF THE INVENTION

1. Field of the Invention

The present invention relates generally to medical methods and devices. More particularly, the present invention relates to methods and systems for delivering agents adjacent to or within the encircling or encapsulating smooth muscle or connective tissue component of a conduit, vessel, or cavitary organ for the prophylaxis or treatment of disease.

Of particular interest to the present invention is the treatment of asthma. The bronchi in the respiratory tract conduct air into the lungs. Smooth muscle is present continuously around the bronchi. Although other factors may be involved, the development of asthma (narrowing of the airways) is most often attributed to hyperreactivity of the smooth muscle of the bronchi accompanied by inflammation. Combined with increased mucus production, intermittent airway obstruction may occur. Some cases of asthma may arise in part from such etiologies as viral or bacterial respiratory infection or chronic allergic processes.

Asthma may be medically managed by the inhalation or oral administration of agents that reduce inflammation and/or relax smooth muscle. However, the time required for onset of relief often limits any acute benefit from such agents, and their chronic use risks favoring selection of agent-resistant populations of microorganisms, which can then lead to perpetuation of inflammation.

A proposed asthma treatment known as bronchial thermoplasty, is described in Cox et al. (2004) Eur Respir J. 4:659-63. In this procedure, a catheter is introduced into a bronchial conduit, and radio-frequency ablation members are brought into contact with the conduit wall to deliver energy sufficient to ablate airway smooth muscle. The ablation of airway smooth muscle, however, is a permanent and damaging procedure that can cause scar tissue formation cannot be reversed.

Other diseases of or around bronchial passageways can cause obstruction or narrowing of the bronchi. Chronic obstructive pulmonary disease (COPD) and cancer are two such causes of narrowing for which medication delivered directly into the wall, whether anti-inflammatory, chemotherapeutic, paralytic, or otherwise may reduce the luminal narrowing and improve airflow without constriction.

In addition to diseases of the respiratory system, diseases of other body lumens, conduits, or cavitary organs can cause obstruction of the conduit, or may simply be best treated by approaching from the conduit and delivering medication directly into the tissue surrounding the conduit. Examples of these diseases include but are not limited to prostate cancer, benign prostatic hyperplasia, esophageal cancer, urethral stricture, bladder cancer, cervical cancer, pancreatic cancer, or biliary obstruction.

For these reasons, it would be desirable to provide improved and alternative treatments for asthma and other luminal diseases. In particular, it would be useful to provide treatment with a rapid onset of action and which minimize long term sequalae of chronic immunosuppression. It would further be desirable to be able to target the treatment to hyperconstrictive bronchial smooth muscle cells of other target tissues without damage or scar tissue creation. At least some of these objectives will be met by the inventions described below.

2. Description of the Background Art

Cox et al. (2004 Eur. Respir. J. 4:659-63 has been described above. Microneedle catheters suitable for intraluminal drug injection are described in U.S. Pat. No. 6,663,821 and U.S. Pat. No. 7,141,041, having a common inventor with the present application, the disclosures of which are incorporated fully herein by reference.

### BRIEF SUMMARY OF THE INVENTION

According to the present invention, therapeutic, diagnostic, or other agents are delivered into smooth muscle or connective tissue surrounding a conduit, vessel or cavitary organ. Delivery is typically accomplished via injection or infusion most often using a needle catheter, usually a microneedle catheter as described in detail below.

The agents are usually therapeutic agents used in a system for treating disease. The agent or agents may also be used for reducing sequelae from a procedure requiring instrumentation of the vessel, conduit, or cavitary organ in question. The treatment period may be of any duration that the physician deems suitable to ensure treatment of the condition under consideration.

### BRIEF DESCRIPTION OF THE DRAWINGS

FIG. 1A is a schematic, perspective view of an intraluminal/intraconduit injection catheter suitable for use in the methods and systems of the present invention.

FIG. 1B is a cross-sectional view along line 1B-1B of FIG. 1A.

FIG. 1C is a cross-sectional view along line 1C-1C of FIG. 1A.

FIG. 2A is a schematic, perspective view of the catheter of FIGS. 1A-1C shown with the injection needle deployed.

FIG. 2B is a cross-sectional view along line 2B-2B of FIG. 2A.

FIG. 3 is a schematic, perspective view of the intraluminal/intraconduit catheter of FIGS. 1A-1C injecting therapeutic agent into an adventitial, extraluminal, or periluminal space surrounding a body lumen in accordance with the methods of the present invention.

FIG. 4 is a schematic, perspective view of another embodiment of an intraluminal/intraconduit injection catheter useful in the methods of the present invention.

FIG. 5 is a schematic, perspective view of still another embodiment of an intraluminal/intraconduit injection catheter useful in the methods of the present invention, as inserted into a body lumen or conduit of a patient.

FIG. 6A and 6B are schematic views of other embodiments of an intraluminal injection catheter useful in the methods of the present invention (in an unactuated condition) including multiple needles.

FIG. 7 is a schematic view of yet another embodiment of an intraluminal injection catheter useful in the methods of the present invention (in an unactuated condition).

FIG. 8 is a perspective view of a needle injection catheter useful in the methods and systems of the present invention.

FIG. 9 is a cross-sectional view of the catheter FIG. 8 shown with the injection needle in a retracted configuration.

FIG. 10 is a cross-sectional view similar to FIG. 9, shown with the injection needle laterally advanced into periluminal tissue for the delivery of drug according to the present invention.

FIG. 11 is a diagram of representative conduits of the human respiratory system, including the bronchi B and trachea T, around which agents may be delivered according to the present invention.

FIG. 12 is a diagram of representative conduits of the male genitourinary system, including the urethra, the prostatic urethra, the bladder, the ureter, and the vas deferens, around which agents may be delivered according to the present invention.

FIG. 13 is a diagram of representative conduits of the female genitourinary system, including the urethra, bladder, ureter, vagina, uterus, fallopian tube, around which agents may be delivered according to the present invention.

FIG. 14 is a diagram of the representative conduits of the gastrointestinal and biliary systems, including the mouth, esophagus, stomach, small intestine (including the duodenum, jejunum and ileum), large intestine (including the cecum, colon and rectum), anus, biliary duct, and gallbladder, around which agents may be delivered according to the present invention.

### DETAILED DESCRIPTION OF THE INVENTION

Subantimicrobial concentrations of anti-infective agents may be used for the purposes of reducing inflammation. The subantimicrobial concentration of any anti-infective agent would be determined via standard laboratory assays, such as minimal inhibitory concentration (MIC). Prior art as to the determination of said concentrations are also described in US RE 34656.

The methods of delivery of an agent in accordance with the present invention may take various forms, but are generally designed to have characteristics appropriate for the intended method of delivery, e.g., through the orifice of a cavitary organ or via puncture through the wall of a conduit or vessel. Injection or infusion using a microneedle catheter is described generally in U.S. patent application Ser. Nos. 09/961,079; 09/961,080; 10/490,129, and 10/490,191 and U.S. Pat. Nos. 6,547,803 and 6,860,867, which describe microneedle catheters and methods of use. U.S. Pat. No. 4,578,061 describes needle injection catheters having deflectable, axially advanceable needles. U.S. Pat. No. 5,538,504 describes a needle injection catheter having a transversely oriented needle that is laterally advanced by a balloon driver. Also of interest are U.S. Pat. Nos. 6,319,230; 6,283,951; 6,283,947; 6,004,295; 5,419,777; and 5,354,279. U.S. patent application Ser. Nos. 10/350,314; 10/610,790; 10/728,186; 10/691,119; 10/393,700; and 10/824,768 are of common invention as this application and describe devices and methods for perivascular (peri-luminal) agent delivery, the entire disclosure of which are incorporated herein by reference.

For purposes of this description, we use the following terms as defined in this section, unless the context of the word indicates a different meaning.

The term “conduit” is meant to refer to a structural element containing a hollow lumen that is intended to transport gaseous, semisolid, or solid materials from one site in the body to another.

The term “vessel” is meant to refer to a structural element containing a hollow lumen that is intended to transport liquid materials from one site in the body to another.

The term “cavitary organ” is mean to refer to an organ containing a space or spaces into which material is produced or transported.

Representative conduits, vessels, and cavitary organs include, but are not limited to the adrenal glands, biliary tree, bladder, bone marrow, brain, bronchi, colon, canal of Schlemm, duodenum, esophagus, eye, Fallopian tubes, gallbladder, heart, hypothalamus, intestine, jejunum, joint spaces, kidneys, liver, lung, lymphatics, lymph nodes, ovaries, pancreas, parathyroids, pituitary, prostate, spinal canal, spleen, stomach, muscle tendon sheath, testes, thyroid, ureters, urethra, uterus, vagina, and vasculature (venous and arterial systems).

The term “subject” is meant to refer to all mammalian subjects, preferably humans. Mammals include, but are not limited to, primates, farm animals, sport animals, cats, dogs, rabbits, mice, and rats.

The terms “treat”, “treating”, or “treatment” are meant to refer to the resolution, reduction, or prevention of disease or the sequelae of disease.

As used herein, the terms “agent” and “drug” are used interchangeably and refer to any substance used for purposes of treatment.

The term “subantimicrobial concentration” is meant to refer to a concentration of anti-infective agent that does not produce toxic effects on or reduction in the growth of the target organism against which it is customarily directed.

The term “anti-infective agents” generally includes antibacterial agents, antifungal agents, antiviral agents, and antiseptics.

Examples of antibacterial agents that may be used at subantimicrobial concentrations include aminoglycosides, amphenicols, ansamycins, lactams, lincosamides, macrolides, nitrofurans, quinolones, sulfonamides, sulfones, tetracyclines, and any of their derivatives. In one variation, tetracyclines are the preferred antibacterial agents. The tetracyclines that may be used include tetracycline itself, doxycycline, and minocycline.

Examples of antifungal agents that may be used at subantimicrobial concentrations include allylamines, imidazoles, polyenes, thiocarbamates, triazoles, and any of their derivatives. In one variation, imidazoles are the preferred antifungal agents.

Other agents possessing anti-inflammatory effects that may be used include, but are not limited to: steroidal agents such as prednisone, methylprednisolone, solumedrol, triamcinolone, betamethasone, and the like; cytokines such as interferon alpha-2a, interferon alpha-2b, interferon beta-1a, interferon beta-1b, interferon gamma, and the like; antibodies such as rituximab, adalimumab, infliximab, alefacept, etanercept, and the like; gamma globulin; statins such as atorvastin, fluvastatin, lovastatin, mevastatin, pravastatin, rosuvastatin, simvastatin, and the like; fenofibrate; gemfibrozil; niacin; niacinamide; nicotine; antihistamines such as diphenhydramine, triprolidine, tripelenamine, fexofenadine, chlorpheniramine, doxylamine, cyproheptadine, meclizine, promethazine, phenyltoloxamine, hydroxyzine, brompheniramine, dimenhydrinate, cetirizine, loratadine, and the like; antidiabetes agents such as acarbose, glimepride, glyburide, metformin, miglitol, pioglitazone, repaglinide, rosiglitazone, and the like; nonsteroidal anti-inflammatory agents such as aspirin, salicylic acid, salsalate, diflunisal, ibuprofen, indomethacin, oxaprozin, sulindac, ketorolac, ketoprofen, nabumetone, piroxicam, naproxen, diclofenac, celecoxib, rofecoxib, valdecoxib, and the like; immunomodulatory agents such as cyclosporine, tacrolimus, pimecrolimus, levamisole, mycophenolate mofetil, methotrexate, cyclophosphamide, azathioprine, hydroxychloroquine, aurothioglucose, auranofin, penicillamine, sulfasalazine, leflunomide, sirolimus, paclitaxel, docetaxel, and the like; beta adrenergic inhibitors such as atenolol, betaxolol, bisoprolol, carvedilol, esmolol, labetalol, metoprolol, nadolol, pindolol, propanolol, sotalol, timolol, and the like; cholinergics such as bethanechol, oxotremorine, methacholine, cevimeline, carbachol, galantamine, arecoline, and the like; muscarine; pilocarpine; anticholinesterases such as edrophonium, neostigmine, donepezil, tacrine, echothiophate, demecarium, diisopropylfluorophosphate, pralidoxime, galanthamine, tetraethyl pyrophosphate, parathion, malathion, isofluorophate, metrifonate, physostigmine, rivastigmine, abenonium acetylchol, carbaryl acetylchol, propoxur acetylchol, aldicarb acetylchol, and the like; calcium channel blockers such as amlodipine, diltiazem, felodiipine, isradipine, nicardipine, nifedipine, nisoldipine, verapamil, and the like; sodium channel blockers such as moricizine, propafenone, encainide, flecainine, tocainide, mexilietine, phenytoin, lidocaine, disopyramine, quinidine, procainamide, and the like; mifepristone; vesicular monoamine transport agents such as guanadrel, guanethidine, reserpine, mecamylamine, hexemethonium, and the like; hydralazine; minoxidil; combination adrenergic inhibitors such as labetalol, carvedilol, and the like; alpha-adrenergic blockers such as doxazosin, prazosin, terazosin, and the like; nitrate derivatives such as L-arginine; nitroglycerine, isosorbide, mononitrate, dinitrate, tetranitrate, and the like; endothelin receptor antagonists such as ambrisentan, bosentan, and the like; phosphodiesterase inhibitors such as vardenafil, tadalafil, sildenafil, and the like; spironolactone, eplerenone, and the like; angiotensin receptor antagonists such as candesartan, irbesartan, losartan, telmisartin, valsartan, eprosartan, and the like; ACE inhibitors such as benazepril, captopril, enalapril, fosinopril, lisinopril, moexipril, quinapril, ramipril, trandolapril, and the like; neurotoxins such as resinoferatoxin, alpha-bungarotoxin, tetrodotoxin, botulinum toxin, and the like; renin inhibitors such as aliskiren, and the like; anticoagulants such as heparin, low molecular weight heparin, fondaparinux, coumadin, acenocoumarol, phenprocoumon, phenindione, argatroban, lepirudin, bivalirudin, clopidogrel, ticlopidine, cilostazol, abciximab, eptifibatide, tirofiban, dipyridamole, and the like; thrombolytic agents such as alteplase, reteplase, urokinase, streptokinase, tenectaplase, lanoteplase, anistreplase, and the like; leukotriene antagonists such as montelukast, zafirlukast, and the like. Such agents may include agents that influence the autonomic nervous system. Such agents include, but are not limited to, beta-blockers, aldosterone antagonists, angiotensin II receptor blockades, angiotensin converting enzyme (“ACE”) inhibitors, endothelin receptor antagonists, sympathomimetics, calcium channel blockers; sodium channel blockers, vasopressin inhibitors, peripheral adrenergic inhibitors; oxytocin inhibitors, botulinum toxin, statins, triglyceride lowering agents, niacin, diabetes agents, immunomodulators, nicotine, sympathomimetics, antihistamines, cholinergics, acetylcholinesterase inhibitors, magnesium and magnesium sulfates, calcium channel blockers, muscarinics, sodium channel blockers, glucocorticoid receptor blockers, blood vessel dilators, central agonists, combined alpha and beta-blockers, alpha blockers, combination diuretics, potassium sparing diuretics, cyclic nucleotide monophosphodiesterase inhibitors, alcohols, vasopressin inhibitors, oxytocin inhibitors, glucagon-like peptide 1, relaxin, renin inhibitors, estrogen and estrogen analogues and metabolites, progesterone inhibitors, testosterone inhibitors, gonadotropin-releasing hormone analogues, gonadotropin-releasing hormone inhibitors, type 4 phosphodiesterase inhibitors, vesicular monoamine transport inhibitors, melatonin, anticoagulants, beta agonists, alpha agonists; indirect agents that include norepinephrine, epinephrine, norepinephrine, acetylcholine, sodium, calcium, angiotensin I, angiotensin II, angiotensin converting enzyme I, angiotensin converting enzyme II, aldosterone, potassium channel blockers and magnesium channel blockers, cocaine, amphetamines, ephedrine, terbutaline, dopamine, dobutamine, antidiuretic hormone, oxytocin, and THC cannabinoids.

Specific autonomic nervous system modulators by name that may be employed in the practice of the subject invention, include, but are not limited to one or more of: beta-blockers: atenolol (e.g., as sold under the brand names Tenormin), betaxolol (e.g., as sold under the brand name Kerlone), bisoprolol (e.g., as sold under the brand name Zebeta), carvedilol (e.g., as sold under the brand name Coreg), esmolol (e.g., as sold under the brand name Brevibloc), labetalol (e.g., as sold under the brand name Normodyne), metoprolol (e.g., as sold under the brand name Lopressor), nadolol (e.g., as sold under the brand name Corgard), pindolol (e.g., as sold under the brand name Visken), propranolol (e.g., as sold under the brand name Inderal), sotalol (e.g., as sold under the brand name Betapace), timolol (e.g., as sold under the brand name Blocadren), carvedilol, and the like; aldosterone antagonists: e.g., spironolactone, eplerenone, and the like; angiotensin II receptor blockades: e.g., candeartan (e.g., available under the brand name Altacand), eprosarten mesylate (e.g., available under the brand name Tevetan), irbesartan (e.g., available under the brand name Avapro), losartan (e.g., available under the brand name Cozaar), etelmisartin (e.g., available under the brand name Micardis), valsartan (e.g., available under the brand name Diovan), and the like; angiotensin converting enzyme (“ACE”) inhibitors: e.g., benazapril (e.g., available under the brand name Lotensin), captopril (e.g., available under the brand name Capoten), enalapril (e.g., available under the brand name Vasotec), fosinopril (e.g., available under the brand name Monopril), lisinopril (e.g., available under the brand name Prinivil), moexipril (e.g., available under the brand name Univasc), quinapril (e.g., available under the brand name AccupriL), ramipril (e.g., available under the brand name Altace), trandolapril (e.g., available under the brand name Mavik), and the like; sympathomimetics: e.g., trimethaphan, clondine, reserpine, guanethidine, and the like; calcium channel blockers: e.g., amlodipine besylate (e.g., available under the brand name Norvasc), diltiazem hydrochloride (e.g., available under the brand names Cardizem CD, Cardizem SR, Dilacor XR, Tiazac), felodipine plendil isradipine (e.g., available under the brand names DynaCirc, DynaCirc CR), nicardipine (e.g., available under the brand name Cardene SR), nifedipine (e.g., available under the brand names Adalat CC, Procardia XL), nisoldipine sulfur (e.g., available under the brand name Sular), verapamil hydrochloride (e.g., available under the brand names Calan SR, Covera HS, Isoptin SR, Verelan) and the like; sodium channel blockers: e.g., moricizine, propafenone, encainide, flecainide, tocainide, mexiletine, phenytoin, lidocaine, disopyramide, quinidine, procainamide, and the like; vasopressin inhibitors: e.g., atosiban (Tractocile), AVP V1a (OPC-21268, SR49059 (Relcovaptan)), V2 (OPC31260, OPC-41061 (Tolvaptan), VPA-985 (Lixivaptan), SR121463, VP-343, FR161282) and mixed V1a/V2 (YM-087 (Conivaptan), JTV-605, CL-385004) receptor antagonists, and the like; peripheral adrenergic inhibitors: e.g., guanadrel (e.g., available under the brand name Hylorel), guanethidine monosulfate (e.g., available under the brand name Ismelin), reserpine (e.g., available under the brand names Serpasil, Mecamylamine, Hexemethonium), and the like; blood vessel dilators: e.g., hydralazine hydrocholoride (e.g., available under the brand name Apresoline), minoxidil (e.g., e.g., available under the brand name Loniten), and the like; central agonists: e.g., alpha methyldopa (e.g., available under the brand name Aldomet), clonidine hydrochloride (e.g., available under the brand name Catapres), guanabenz acetate (e.g., available under the brand name Wytensin), guanfacine hydrochloride (e.g., available under the brand name Tenex), and the like; combined alpha and beta-blockers: e.g., carvedilol (e.g., available under the brand name Coreg), labetolol hydrochloride (e.g., available under the brand names Normodyne, Trandate), and the like; alpha blockers: e.g., doxazosin mesylate (e.g., available under the brand name Cardura), prazosin hydrochloride (e.g., available under the brand name Minipress), terazosin hydrochloride (e.g., available under the brand name Hytrin), and the like; renin inhibitors: e.g., Aliskiren, and the like; oxytocin inhibitors: e.g., terbutaline, ritodrine, and the like, and botulism toxin (or botox) and the like.

Other potential agents include smooth muscle relaxants that may include, but are not limited to, alvarine, anisotropine, atropine, belladonna, clidinium, dicyclomine, glycopyrrolate, homatropine, hyoscyamine, mebevarine, mepenzolate, methantheline, methscopolamine, oxybutynin, papavarine, pirenzepine, popantheline, scopolamine, and the like.

Other potential agents include chemotherapeutic agents, specifically those cytotoxic agents traditionally used to treat cancer. Such agents may include, but are not limited to, alkylating agents such as busulfan, hexamethylmelamine, thiotepa, cyclophosphamide, mechlorethamine, uramustine, melphalan, chlorambucil, carmustine, streptozocin, dacarbazine, temozolomide, ifosfamide, and the like; anti-metabolites such as methotrexate, azathioprine, mercaptopurine, fludarabine, 5-fluorouracial, and the like; anthracyclines such as daunorubicin, doxorubicin, epirubicin, idarubicin, mitoxantrone, and the like; plant alkaloids and terpenoids such as vincristine, vinblastine, vinorelbine, vindesine, podophyllotoxin, paclitaxel, doclitaxel, and the like; topoisomerase inhibitors such as irinotecan, amsacrine, topotecan, etoposide, teniposide, and the like; antibody agents, such as rituximab, trastuzumab, bevacizumab, erlotinib, dactinomycin; finasteride; aromatase inhibitors; tamoxifen; goserelin; imatinib mesylate.

The variations of this invention may further include components such as preservatives, buffers, binders, disintegrants, lubricants, and any other excipients necessary to maintain the structure and/or function of the anti-infective agents.

Furthermore, the agents may be placed in a pharmaceutically acceptable carrier for purposes of delivery. Common bases include, but are not limited to, carbomer, liquid paraffin, water, glycerol, propylene glycol, polyethylene glycol, sodium corboxymethylcellulose, polysorbate (polyoxyethylene sorbitan monooleate), dimethyl sulfoxide, dimethylacetamide, ethanol, benzyl alcohol, hyaluronic acid or sodium hyaluronate, or a combination thereof.

The agent or agents may be used to treat a condition including, but not limited to, cardiovascular diseases, such as carotid aneurysm, carotid obstruction, atherosclerosis, coronary artery disease, hypertension, hyperlipidemia, eclampsia, pre-eclampsia, cardiomyopathy, volume retention, congestive heart failure, QT interval prolongation, aortic dissection, aortic aneurysm, arterial aneurysm, arterial vasospasm, myocardial infarction, reperfusion syndrome, ischemia, sudden adult death syndrome, fatal arrhythmias, coronary syndromes, coronary vasospasm, sick sinus syndrome, bradycardia, tachycardia, arrhythmias, thromboembolic disease, deep vein thrombosis, coagulopathy, DIC, mesenteric ischemia, syncope, venous thrombosis, arterial thrombosis, malignant hypertension, secondary hypertension, primary pulmonary hypertension, secondary pulmonary hypertension, Raynaud's syndrome, paroxysmal supraventricular tachycardia, and the like; neurodegenerative diseases, such as Alzheimer's disease, Pick's disease, Parkinson's disease, amyotrophic lateral sclerosis, an d the like; neuroinflammatory diseases, such as headache, migraine, viral meningitis, bacterial meningitis, viral encephalitis, fungal meningitis, fungal encephalitis, multiple sclerosis, schizophrenia, and the like; orthopedic inflammatory diseases, such as osteoarthritis, reflex sympathetic dystrophy, osteoporosis, regional idiopathic osteoporosis, Paget's disease, juvenile chronic arthritis, antigen-induced arthritis, rotator cuff syndrome, tendonitis, and the like; hematologic diseases, such as aplastic anemia, red cell aplasia, lymphoma, lymphoproliferative disease, myelodysplastic syndrome, leukemia, Hodgkin's disease, inflammatory pseudotumor of the liver, and the like; autoimmune diseases, such as Graves' disease, Hashimoto's disease, Takayasu's disease, Kawasaki's disease, arteritis, scleroderma, CREST syndrome, allergies, dermatitis, Henoch-Schlonlein purpura, Goodpasture's Syndrome, autoimmune thyroiditis, myasthenia gravis, lupus, Reiter's Syndrome, Sjogren's syndrome, sarcoidosis, and the like; inflammatory and infectious diseases, such as sepsis, diseases of wound healing, cellulitis, bacterial infection, viral infection, tuberculosis, fungal infection, human immunodeficiency virus infection and the like; pulmonary diseases, such as asthma, reactive airway disease, tachypnea, fibrotic lung diseases such as idiopathic pulmonary fibrosis and asbestosis, cystic fibrosis, interstitial lung disease, chemical pneumonitis, desquamative interstitial pneumonitis, non-specific interstitial pneumonitis, lymphocytic interstitial pneumonitis, usual interstitial pneumonitis, idiopathic pulmonary fibrosis, pulmonary edema, aspiration, asphyxiation, pneumothorax, right-to-left shunts, left-toright shunts, respiratory failure, pneumonia, chronic obstructive pulmonary disease, emphysema, bronchitis, and the like; gastrointestinal disorders, such as cirrhosis, xerostomia, bowel mobility, constipation, irritable bowel syndrome, primary biliary cirrhosis, viral hepatitis, chemical hepatitis, fatty liver, ileus, post-operative bowel dysmotility, cholelithiasis, cholestasis, cholecystitis, sclerosing cholangitis, biliary strictures, fecal incontinence, cyclic vomiting syndrome, diverticulitis/diverticulosis, ulcerative colitis, Crohn's disease, celiac sprue, tropical sprue, infectious diarrhea, noninfectious diarrhea, constipation, esophagitis, gastroesophageal reflux, gastritis, peptic ulcer disease, and the like; endocrine disorders, such as hypothyroidism, hyperthyroidism, diabetes, obesity, syndrome X, hyperglycemia, insulin resistance, adrenal hyperplasia, adrenal insufficiency, adrenal inflammation, and the like; genitourinary disorders, such as cystitis, bladder dysfunction, renal failure, erectile dysfunction, prostatitis, erectile dysfunction, benign prostatic hypertrophy, hyperreninemia, hepatorenal syndrome, pulmonary renal syndrome, incontinence, arousal disorders, menopausal mood disorders, premenstrual mood disorders, orchitis, vaginitis, urethritis, and the like; skin disorders, such as wrinkling, atopic dermatitis, psoriasis, lichen planus, allergic dermatitis, cutaneous vasculitis, and the like; aging associated diseases and conditions, such as Shy-Drager's, multi-symptom atrophy, age related inflammatory conditions, cancer, aging and the like; conditions that cause hypoxia, hypercarbia, and/or acidosis; sudden infant death syndrome, sudden adult death syndrome, acute pulmonary embolism, chronic pulmonary embolism, pleural effusion, cardiogenic pulmonary edema, non-cardiogenic pulmonary edema, acute respiratory distress syndrome (“ARDS”), neurogenic edema, acidosis of any cause, hypercapnia, acidemia, renal tubular acidosis, asthma, any chronic lung disease that causes hypoxia or hypercarbia or hypercapnia, and the like; neurologic diseases, such as epilepsy, moya/moya, seizures, stroke, insomnia, sleep disorders, transient ischemic attacks, headaches, concussions, post-concussive syndrome, cerebral aneurysm, cerebrovascular vasospasm, central sleep apnea, obstructive sleep apnea, stress, bipolar disorder, depression, and the like; pediatric conditions, e.g., respiratory distress syndrome, sudden infant death syndrome, Hirschsprung's disease, bronchopulmonary dysplasia, congenital megacolon, aganglionosis, juvenile rheumatoid arthritis, and the like; obstetric/gynecologic diseases, e.g., amniotic fluid embolism, pregnancy-related arrhythmias, fetal stress, fetal hypoxia, amniotic fluid embolism, gestational diabetes, pre-term labor, cervical incompetence, fetal distress, peri-partum maternal mortality, labor complications, premenstrual syndrome, dysmenorrhea, endometriosis, fibroids, dyspareunia, polycystic ovary disease and the like; as well as other conditions, including but not limited to: chronic pain, glaucoma, trauma, hospitalization, post-operative recovery, post-procedural recovery, transplant-related side effects, fibrosis, transplant-related tachycardia, transplant rejection, transplant-related bowel dysmotility, transplant-related hyperreninemia, male infertility, disorders of thermoregulation, fibromyalgia, and the like; ophthalmic diseases, such as glaucoma, macular degeneration, cataracts, retinopathy, and the like; venous thrombosis, peripheral arterial disease, as well as any malignancies involving the aforementioned roster of vessels, conduits, and cavitary organs. Such malignancies may include, but are not limited to, bladder cancer, bone cancer, breast cancer, brain cancer, leukemia, lymphoma, colon cancer, esophageal cancer, gallbladder cancer, intestinal cancer, renal cell carcinoma, hepatocarcinoma, lung cancer, ovarian cancer, pancreatic cancer, prostate cancer, parathyroid cancer, stomach cancer, testicular cancer, thyroid cancer, uterine cancer.

Furthermore, the agents may be used to treat acute or chronic inflammation in vessels, conduits, and cavitary organs, arising from predisposing anatomical conditions, chronic allergic processes, or conditions related to infection by various pathogens (e.g., bacteria, fungi, and viruses).

The agents may also be used to reduce inflammation resulting from a procedure. Examples of procedures include, but are not limited to, widening/enlargement of a narrowed lumen or orifice, puncture and evacuation or washout of a vessel, conduit, or cavitary organ, and creation of a novel lumen or orifice within or adjacent to an extant vessel, conduit, or cavitary organ. The agents may be delivered into the region in question after the procedure is completed, but they can also be delivered before the procedure or during the procedure.

The present invention will preferably utilize microfabricated devices and methods for periluminal delivery of agents. The following description provides several representative embodiments of microfabricated needles (microneedles) and macroneedles suitable for the delivery of the agent into a periluminal (to tissue surrounding the vessel, conduit or cavitary organ) tissue. The periluminal tissue is the tissue outside the border defined by the boundary between the “lumen” and “lumen wall” of a vessel, conduit or cavitary organ. The microneedle is usually inserted substantially normal to the wall of the lumen to eliminate as much trauma to the patient as possible, though this is not a requirement and often depends on the orientation and anatomical variation of the lumen. Until the microneedle or microports are at the site of an injection or infusion, they are positioned in an atraumatic configuration so that there is no scraping against the wall of the lumen by any tip of a needle or port structure. In one specific embodiment, a microneedle remains enclosed in the walls of an actuator or sheath attached to a catheter so that it will not injure the patient during intervention or the physician during handling. When the injection site is reached, movement of the actuator along the lumen is terminated, and the actuator is operated to cause the microneedle to be thrust outwardly, substantially perpendicular to the central axis of a lumen, for instance, in which the catheter has been inserted.

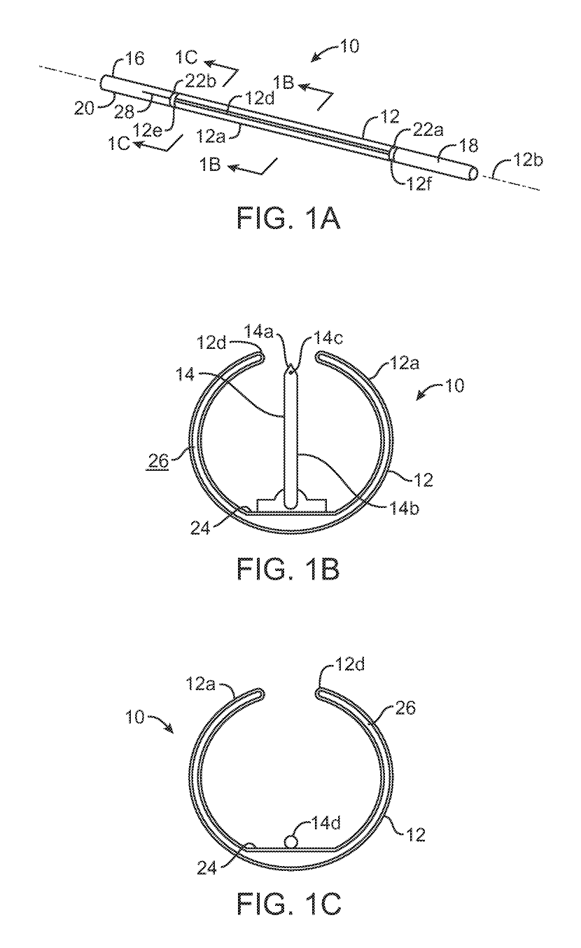

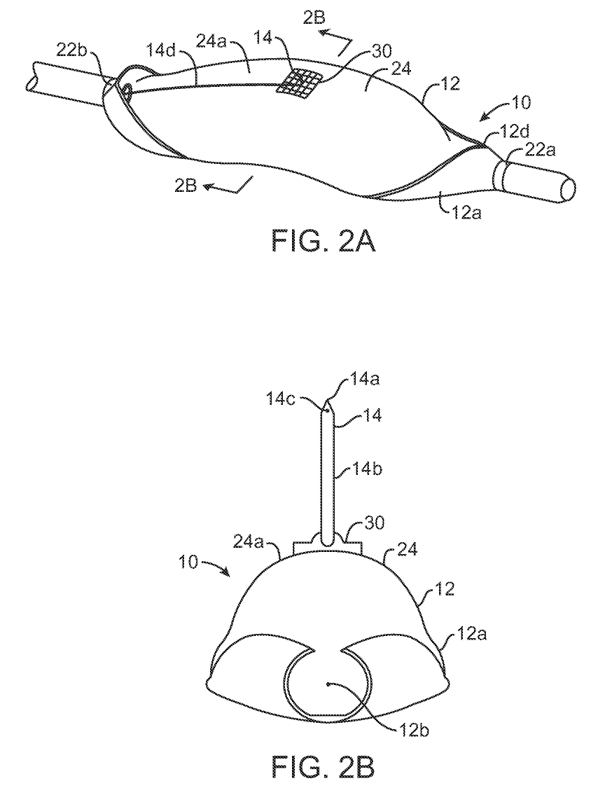

As shown in FIGS. 1A-2B, a microfabricated intra-luminal catheter 10 suitable for use in the methods of the present invention is described in U.S. Pat. No. 6,547,803, and includes an actuator 12 having an actuator body 12a and central longitudinal axis 12b. The actuator body more or less forms a U-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a target sinus or other body lumen by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b may be located at the distal and proximal ends, respectively, of the actuator, though their presence is not necessary for appropriate actuation given ideal or near-ideal rigidity of the actuator material. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings or simple rigidity of the structure by itself forms a rigid substantially “C” or “U”—shaped structure at each end and in the center of the actuator. The catheter may be joined to the retaining rings by, for example, a butt-weld, an ultra sonic weld, integral polymer encapsulation or an adhesive such as an epoxy or cyanoacrylate.

The actuator body further comprises a central, expandable section 24 located between the rigid ends or retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 psi upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflon© or other inert plastics. The activating fluid may be a saline solution, a radio-opaque dye, or some combination of the two.

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate.

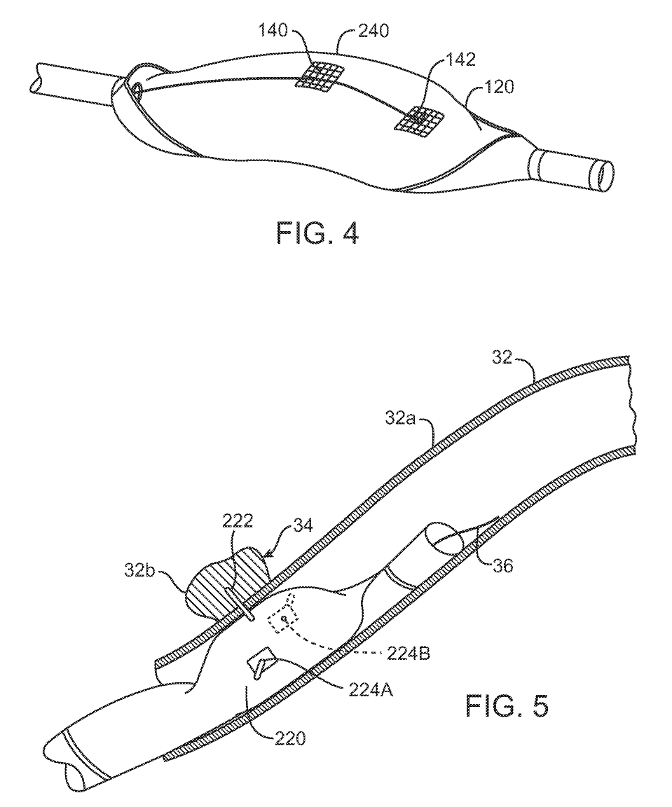

Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon. The microneedle may alternatively be affixed to a tube which is otherwise adhered to the surface 24a by adhesive, encapsulation bonding, or is simply a feature of the surface 24a.

The microneedle includes a sharp tip 14a and a shaft 14b. The microneedle tip can provide an insertion edge or point. The shaft 14b can be hollow and the tip can have an outlet port 14c, permitting the injection of the agent into the sub-epithelial or peri-luminal tissues.

As shown, the microneedle extends approximately perpendicularly from surface 24a. Thus, as described, the microneedle will move substantially perpendicularly to an axis of a lumen into which has been inserted, to allow direct puncture or breach of tissue walls surrounding the lumen, such as the epithelium and paranasal sinus mucosa.

The microneedle further includes a pharmaceutical or drug supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass.

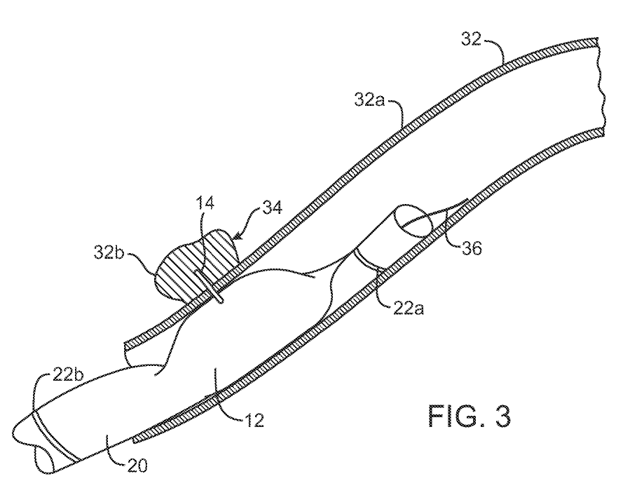

The catheter 20, in use, is inserted into a patient's body lumens, for instance, through a nostril into a paranasal sinus ostium 32, until a specific, targeted region 34 is reached (see FIG. 3). The targeted region 34 may be at or proximate to the site of tissue damage or inflammation, typically being within 100 mm or less to allow migration of the therapeutic agents. As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire. The catheter may instead be inserted under the aid of endoscopic guidance, using a floppy-tipped catheter to minimize trauma.

During maneuvering of the catheter 20, well-known methods of fluoroscopy, endoscopy, or magnetic resonance imaging (MRI) can be used to image the catheter and assist in positioning the actuator 12 and the microneedle 14 at the target region. As the catheter is guided inside the patient's body, the microneedle remains unfurled or held inside the actuator body so that no trauma is caused to the body lumen walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and five seconds for the microneedle to move from its furled state to its unfurled state.

The ends of the actuator at the retaining rings or rigid end conditions 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels with such force that it can enter sub-epithelial or peri-luminal tissue 32b as well as mucosal, or luminal tissue. Additionally, since the actuator is “parked” or stopped prior to actuation, more precise placement and control over penetration of the lumen wall are obtained.

After actuation of the microneedle and delivery of the drugs to the target region via the microneedle, the activating fluid is exhausted from the open area 26 of the actuator, causing the expandable section 24 to return to its original, furled state. This also causes the microneedle to be withdrawn from the lumen wall. The microneedle, being withdrawn, is once again sheathed by the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include imaging components, such as an intravascular ultrasonic sensor (IVUS), for locating lumen walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 μm, while the tube's and shaft's exterior cross-sectional dimension may be between about 100 and 500 μm. The overall length of the actuator body may be between about 3 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 100-500 μm. The diameter of the delivery tube for the activating fluid may be about 100 μm. The catheter size may be between 1.5 and 15 French (Fr).

As shown in FIG. 4, the actuator 120 may include a plurality of microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t1, and pressure p1, and the microneedle 142 moving at time t2 and p2, with t1, and p1, being less than t2 and p2, respectively.

This sort of staged buckling can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a lumen wall. A needle 224B (as shown in phantom) could alternatively be arranged to move at angle of about 180° to the needle 224A.

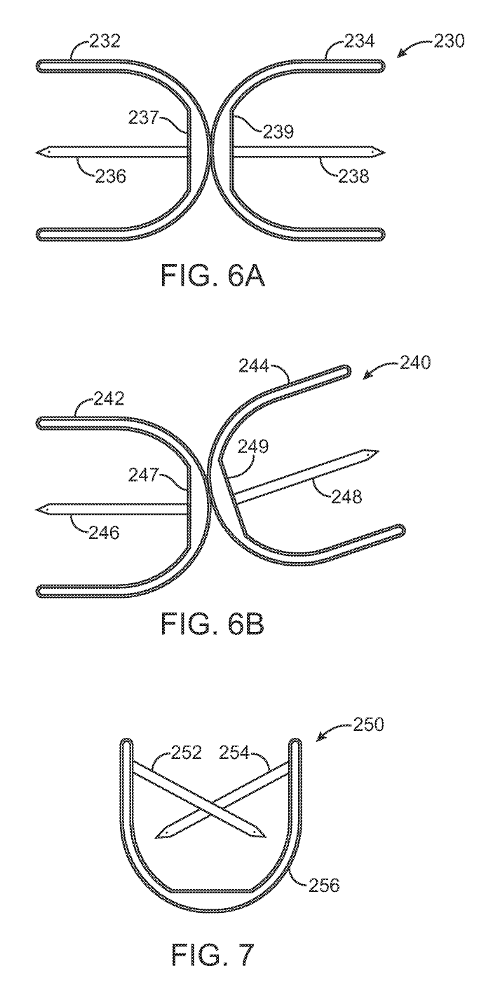

Moreover, as shown in FIG. 6, in another embodiment, an actuator 230 comprises actuator bodies 232 and 234 including needles 236 and 238, respectively, that move approximately horizontally at angle of about 180° to each other. Also, as shown in FIG. 6B, an actuator 240 comprises actuator bodies 242 and 244 including needles 242 and 244, respectively, that are configured to move at some angle relative to each other than 90° or 180°. The central expandable section of the actuator 230 is provided by central expandable sections 237 and 239 of the actuator bodies 232 and 234, respectively. Similarly, the central expandable section of the actuator 240 is provided by central expandable sections 247 and 249 of the actuator bodies 242 and 244, respectively.

Additionally, as shown in FIG. 7, an actuator 250 may be constructed that includes multiple needles 252 and 254 that move in different directions when the actuator is caused to change from the unactuated to the actuated condition. The needles 252 and 254, upon activation, do not move in a substantially perpendicular direction relative to the longitudinal axis of the actuator body 256.

The above catheter designs and variations thereon, are described in published U.S. Patent Application Nos. 2003/005546 and 2003/0055400, the full disclosures of which are incorporated herein by reference. Co-pending application Ser. No. 10/350,314, assigned to the assignee of the present application, describes the ability of substances delivered by direct injection into the adventitial and pericardial tissues of the heart to rapidly and evenly distribute within the heart tissues, even to locations remote from the site of injection. The full disclosure of that co-pending application is also incorporated herein by reference. An alternative needle catheter design suitable for delivering the drug of the present invention will be described below. That particular catheter design is described and claimed in co-pending application Ser. No. 10/393,700 (Attorney Docket No. 021621-001500 U.S.), filed on Mar. 19, 2003, the full disclosure of which is incorporated herein by reference.

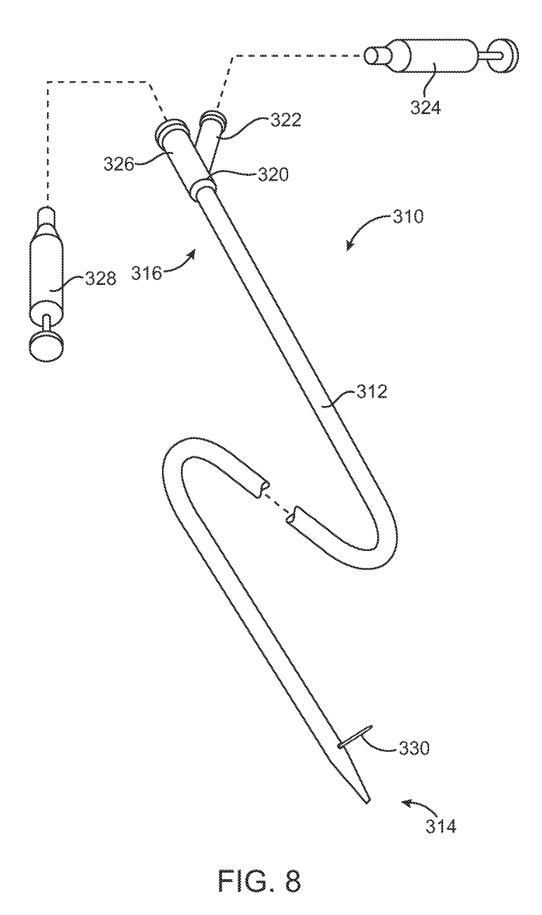

Referring now to FIG. 8, a needle injection catheter 310 constructed in accordance with the principles of the present invention comprises a catheter body 312 having a distal end 314 and a proximal 316. Usually, a guide wire lumen 313 will be provided in a distal nose 352 of the catheter, although over-the-wire and embodiments which do not require guide wire placement will also be within the scope of the present invention. A two-port hub 320 is attached to the proximal end 316 of the catheter body 312 and includes a first port 322 for delivery of a hydraulic fluid, e.g., using a syringe 324, and a second port 326 for delivering the pharmaceutical agent, e.g., using a syringe 328. A reciprocatable, deflectable needle 330 is mounted near the distal end of the catheter body 312 and is shown in its laterally advanced configuration in FIG. 8.

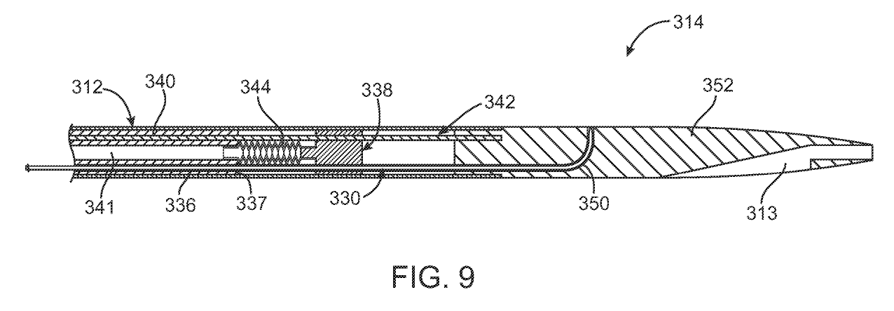

Referring now to FIG. 9, the proximal end 314 of the catheter body 312 has a main lumen 336 which holds the needle 330, a reciprocatable piston 338, and a hydraulic fluid delivery tube 340. The piston 338 is mounted to slide over a rail 342 and is fixedly attached to the needle 330. Thus, by delivering a pressurized hydraulic fluid through a lumen 341 tube 340 into a bellows structure 344, the piston 338 may be advanced axially toward the distal tip in order to cause the needle to pass through a deflection path 350 formed in a catheter nose 352.

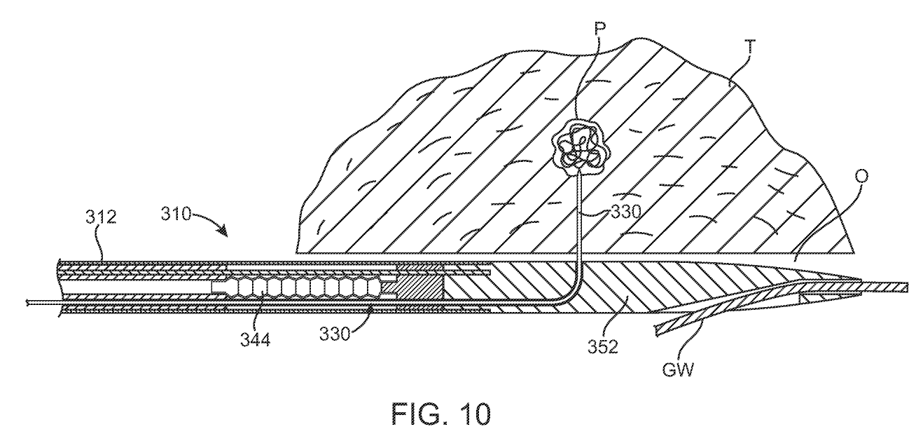

As can be seen in FIG. 10, the catheter 310 may be positioned in a body lumen, conduit or cavitary opening O, over a guide wire GW in a conventional manner. Distal advancement of the piston 338 causes the needle 330 to advance into sub-epithelial or peri-luminal tissue T adjacent to the catheter when it is present in the sinus. The drug may then be introduced through the port 326 using syringe 328 in order to introduce a plume P of drug in the peri-luminal tissue, as illustrated in FIG. 10.

The needle 330 may extend the entire length of the catheter body 312 or, more usually, will extend only partially in drug delivery lumen 337 in the tube 340. A proximal end of the needle can form a sliding seal with the lumen 337 to permit pressurized delivery of the drug through the needle.

The needle 330 will be composed of an elastic material, typically an elastic or super-elastic metal, typically being nitinol or other super elastic metal. Alternatively, the needle 330 could be formed from a non-elastically deformable or malleable metal which is shaped as it passes through a deflection path. The use of non-elastically deformable metals, however, is less preferred since such metals will generally not retain their straightened configuration after they pass through the deflection path.

The bellows structure 344 may be made by depositing by parylene or another conformal polymer layer onto a mandrel and then dissolving the mandrel from within the polymer shell structure. Alternatively, the bellows 344 could be made from an elastomeric material to form a balloon structure. In a still further alternative, a spring structure can be utilized in, on, or over the bellows in order to drive the bellows to a closed position in the absence of pressurized hydraulic fluid therein.

After the drug is delivered through the needle 330, as shown in FIG. 10, the needle is retracted and the catheter either repositioned for further agent delivery or withdrawn. In some embodiments, the needle will be retracted simply by aspirating the hydraulic fluid from the bellows 344. In other embodiments, needle retraction may be assisted by a return spring, e.g., locked between a distal face of the piston 338 and a proximal wall of the distal tip 352 (not shown) and/or by a pull wire attached to the piston and running through lumen 341.

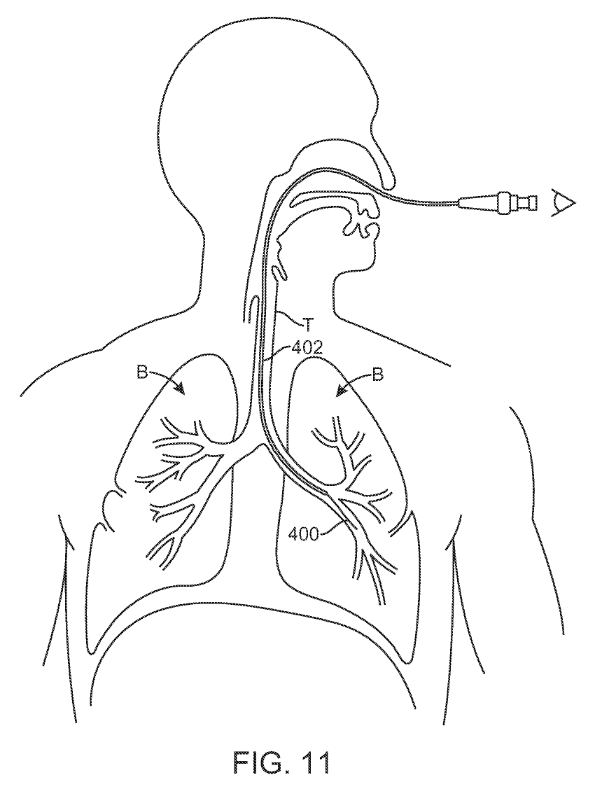

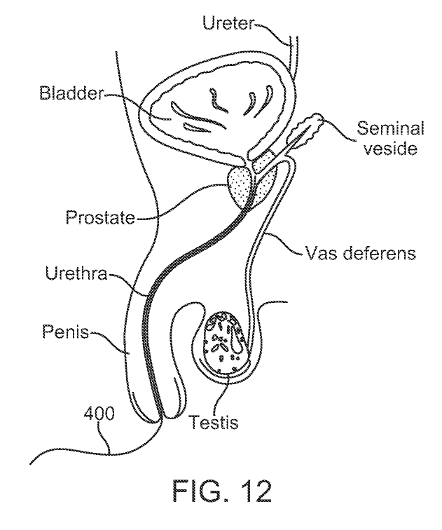

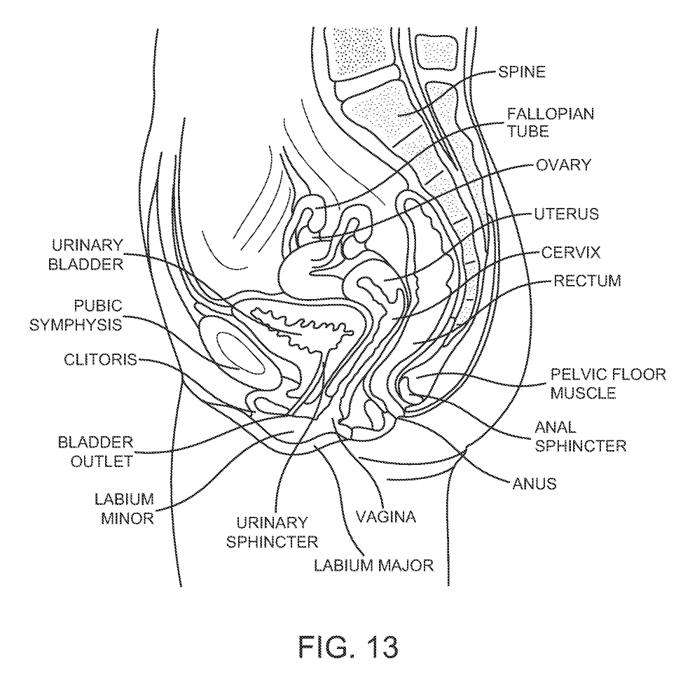

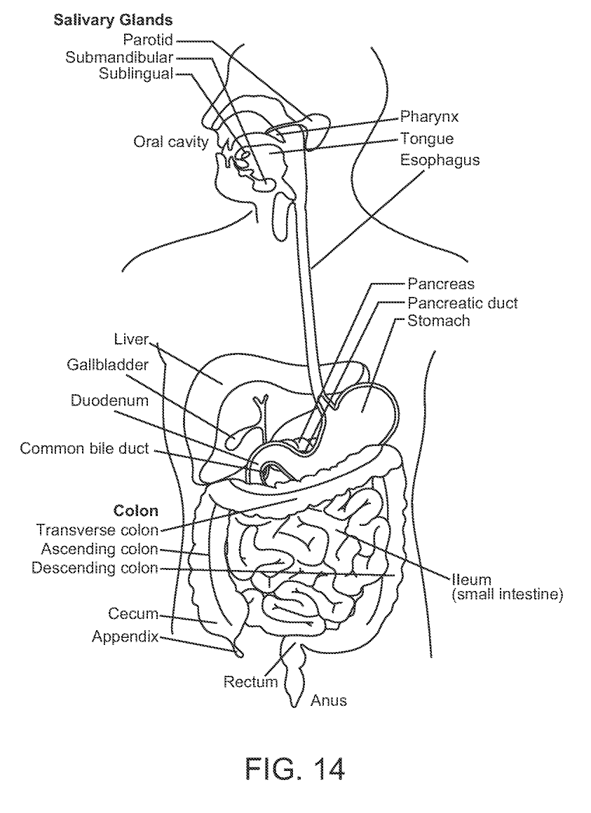

Referring now to FIG. 10 through FIG. 14, body lumens, conduits, vessels, and cavitary organs that may be treated in accordance with the present invention are present in the respiratory system, the male and female genitourinary systems, the gastrointestinal system, and the cardiovascular and lymphatic systems (not depicted in a figure). In each of these systems, a catheter 400 may be introduced to an area of therapeutic interest as described in the text above. At that position, a needle is deployed through the wall of the conduit and medication is delivered. Of particular interest to this invention, medication may be deployed to reduce hyperconstrictive smooth muscle in the lungs, for example in asthmatic patients, where the catheter is typically delivered through a bronchoscope 402 (FIG. 11); medication may be delivered into the prostate via a transurethral catheterization in order to debulk hypertrophic prostate, whether benign or malignant (FIG. 12); anti-cancer therapeutic agents may be delivered into tumors that lie near or around the conduit through which the catheter may be introduced and deployed (i.e. in bladder, lung, or outside the biliary tract; urethral constriction may be treated with peri-urethral delivery of anti-inflammatory or anti-proliferative medication (FIGS. 12 and 13); or agents may be delivered just outside the esophageal conduit to bulk the esophageal sphincter or debulk esophageal cancers (FIG. 14). Additional applications are expanded upon in the examples that follow.

1. Use of Microneedle Catheter to Treat Asthma.

In a double-blind randomized controlled trial, emergency room patients undergoing asthma exacerbation as defined by FEV<60% on admission are assigned to one of two arms. A microneedle catheter is used to inject a proprietary agent into the bronchial smooth muscle of patients in the treatment group; the control group receives standard therapy using bronchodilators and oral steroids. Percentage of responders and response time are significantly greater for the group treated using the microneedle catheter as compared to the control group.

2. Use of Microneedle Catheter to Treat Interstitial Cystitis.

In a double-blind randomized controlled trial, emergency room patients undergoing an exacerbation of interstitial cystitis, as defined by pelvic pain, urinary frequency, and pain with urination with a negative urinalysis, are assigned to one of two arms. A microneedle catheter is used to inject a proprietary agent into the bladder smooth muscle of patients in the treatment group; the control group receives standard rescue therapy using an instillation of heparin, sodium hyaluronate, lidocaine, and sodium bicarbonate. Percentage of responders and response time are significantly greater for patients treated using the microneedle catheter as compared to the control group.

3. Use of Microneedle Catheter to Treat Glaucoma.

In a double-blind randomized controlled trial, ambulatory patients with primary open angle glaucoma and no significant comorbidities are assigned to one of two arms. A microneedle catheter is used to inject a proprietary agent into the wall of the canal of Schlemm of patients in the treatment group; the control group receives laser trabeculoplasty. Extent of lowering of intraocular pressure and overall response rate is significantly greater for the group treated with the microneedle catheter as compared to the control group.

4. Use of Microneedle Catheter to Treat Pulmonary Arterial Hypertension.

In a double-blind randomized controlled trial, patients with significant pulmonary arterial hypertension are assigned to one of two arms. A microneedle catheter is used to inject a proprietary agent into the wall of the artery and its tributary vessels in the treatment group; the control group receives treatment with oral bosentan. Overall response rate as measured by degree of hypertension as well as quality of life indices is significantly greater for the group treated with the microneedle catheter as compared to the control group.

5. Use of Microneedle Catheter to Treat Benign Prostatic Hypertrophy.

In a double-blind randomized controlled trial, patients with benign prostate hypertrophy and no significant comorbidities are assigned to one of two arms. A microneedle catheter is used to inject a proprietary agent into the wall of the prostate in the treatment group; the control group receives conventional therapy with oral finasteride. Overall response rate as measured by reduction of prostatic volume with relief of symptoms is significantly greater for the group treated with the microneedle catheter as compared to the control group.

6. Use of Microneedle Catheter to Treat Cerebral Aneurysm.

In a double-blind randomized controlled trial, patients with uncomplicated cerebral aneurysm as detected by arteriogram with no other significant comorbidities are assigned to one of two arms. A microneedle catheter is used to inject a proprietary agent into the wall of the aneurysm in the treatment group; the control group is treated via coil placement. Rate of aneurismal expansion and overall incidence of rupture is significantly lower for the group treated with the microneedle catheter as compared to the control group.

All publications, patents, and patent applications cited herein are hereby incorporated by reference in their entirety for all purposes to the same extent as if each individual publication, patent, or patent application are specifically and individually indicated to be so incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

What is claimed is:

1. A method for treating or preventing disease, said method comprising delivering at least one agent to smooth muscle or connective tissue which surround a conduit, vessel or cavitary organ.

2. The method of claim 1 wherein the conduits are the bronchi.

3. The method of claim 1 wherein the vessel is the canal of Schlemm.

4. The method of claim 1 wherein the vessel is the pulmonary artery.

5. The method of claim 1 wherein the vessel is an intracerebral artery.

6. The method of claim 1 wherein the cavitary organ is the bladder.

7. The method of claim 1 wherein the cavitary organ is the prostate.

8. The method of claim 1 wherein the disease being treated is asthma.

9. The method of claim 1 wherein the disease being treated is reactive airway disease.

10. The method of claim 1 wherein the disease being treated is glaucoma.

11. The method of claim 1 wherein the disease being treated is pulmonary arterial hypertension.

12. The method of claim 1 wherein the disease being treated is cerebral aneurysm.

13. The method of claim 1 wherein the disease being treated is interstitial cystitis.

14. The method of claim 1 wherein the disease being treated is benign prostatic hypertrophy.

15. The method of claim 1 wherein the agent is delivered into the smooth muscle or connective tissue.

16. The method of claim 1 wherein the agent is delivered to a location adjacent to the smooth muscle or connective tissue.

17. The method of claim 1 wherein the agent is selected from the group consisting of agents modulating the autonomic nervous system, chemotherapeutic agents, and agents with anti-inflammatory activity, the latter including antimicrobial agents used at submicrobial concentrations, including antibacterial agents, antifungal agents, antiviral agents, and antiseptics.

18. The method of claim 17 wherein the agent is triamcinolone.

19. The method of claim 17 wherein the agent is lidocaine.

20. The method of claim 17 wherein the agent is botulinum toxin.

21. The method of claim 17 wherein the agent is paclitaxel.

22. The method of claim 17 wherein the agent is a beta-blocker.

23. The method of claim 22 wherein the beta-blocker is selected from the group consisting of atenolol, betaxolol, bisoprolol, carvedilol, esmolol, labetalol, metoprolol, nadolol, pindolol, propanolol, sotalol, timolol, and any of their derivatives.

24. The method of claim 17 wherein the agent is a statin.

25. The method of claim 24 wherein the statin is selected from the group consisting of atorvastin, fluvastatin, lovastatin, mevastatin, pravastatin, rosuvastatin, simvastatin, and any of their derivatives.

26. The method of claim 17 wherein the anti-infective agent comprises an antibacterial agent.

27. The method of claim 26 wherein the antibacterial agent is selected from the group consisting of aminoglycosides, amphenicols, ansamycins, (3-lactams, lincosamides, macrolides, nitrofurans, quinolones, sulfonamides, sulfones, tetracyclines, and any of their derivatives.

28. The method of claim 27 wherein the antibacterial agent comprises a tetracycline.

29. The method of claim 28 wherein the tetracycline comprises doxycycline.

30. The method of claim 29 wherein doxycycline is administered at a concentration such that local tissue concentrations are obtained which are identical to those achieved with the administration of 20 mg oral equivalent twice a day or less.

31. The method of claim 1 wherein the agent comprises a pharmaceutically acceptable carrier.

32. The method of claim 1 wherein delivering comprises advancing a needle from a catheter disposed in the conduit vessel, or cavitary organ.

33. The method of claim 1 further comprising enlarging the conduit, vessel, or lumen prior to delivering said agent.

34. The method of claim 1 further comprising evacuation and washout of the vessel, conduit, or cavitary organ prior to delivering said agent.

35. The method of claim 1 further comprising draining said vessel, conduit, or organ prior to delivering said agent.

36. The method of claim 1 further comprising creating a lumen or orifice within or adjacent to said vessel, conduit, or cavitary organ prior to delivering said agent.
